(19) United States
(12) Patent Application Publication
(10) Pub. No.: US 2022/0390335 A1
(43) Pub. Date: Dec. 8, 2022

(54) A Multi-Layered Membrane And A Method Of Preparing The Same

(71) Applicant: National University of Singapore, Singapore (SG)

(72) Inventors: Jie Gao, Singapore (SG); Tai-Shung Chung, Singapore (SG); Kwee Hiang Jackson Low, Singapore (SG); Chester Lee Drum, Singapore (SG); E Shyong Tai, Singapore (SG)

(73) Assignee: National University of Singapore, Singapore (SG)

(21) Appl. No.: 17/776,453

(22) PCT Filed: Nov. 12, 2020
(86) PCT No.: PCT/SG2020/050650

§ 371 Date: May 12, 2022

(30) Foreign Application Priority Data
Nov. 12, 2019 (SG) 10201910574R

(51) Int. Cl. G01N 1/40; B01D 69/02; B01D 71/42; B01D 67/00

(52) U.S. Cl. CPC G01N 1/4005; B01D 69/02; B01D 71/421; B01D 67/0013; B01D 67/0018; B01D 67/0088; B01D 67/00933; G01N 2001/4088; B01D 2323/12; B01D 2323/21825; B01D 2323/39; B01D 2323/219; B01D 2325/12; B01D 2325/02834; B01D 2325/022

(57) ABSTRACT

There is provided a multi-layered membrane for separating components in an aqueous sample. There is also provided a method of preparing said multi-layered membrane, a method of separating blood plasma from a whole blood sample and a diagnostic device for separation of blood plasma from a whole blood sample.

### REFERENCES TO RELATED APPLICATIONS

This application is a §371 National Stage Entry of International Application Serial No. PCT/SG2020/050650, filed on 12 Nov. 2020, which claims priority to Singapore Application No. 10201910574R, filed on 12 Nov. 2019, the entire disclosures of which are hereby incorporated by reference.

### TECHNICAL FIELD

The present invention generally relates to a multi-layered membrane for separating components in an aqueous sample. The present invention also relates to a method of preparing the multi-layered membrane. The present invention further relates to a method of separating blood plasma from a whole blood sample. The present invention further relates to a diagnostic device for separation of blood plasma from a whole blood sample.

### BACKGROUND ART

Dried blood spot (DBS) is a minimally invasive blood sampling technique where a few drops of capillary whole blood are collected from a finger prick remotely on a filter paper, dried and transported for future analyses. With the deployment of a valid DB S flow, it will be a much simpler and cheaper process for the public to collect their own biosamples at home and send them for central analyses at national hospitals. Historically, DBS has dealt with dried whole blood on simple cellulose papers.

DBS has advantages over the conventional venous blood collection. It offers a simpler sample collection, storage, and transfer, with reduced infection risk of various pathogens. Moreover, DBS collection is relatively painless and is better suited for patients with damaged/altered veins, the elderly or infants. The use of DBS also minimizes the volume of blood taken from patients. Though DBS is a feasible and widely used blood sampling technique, there are also limitations of DBS. Firstly, the small sample size and assay sensitivity of DBS mean that it needs a high sample quality to provide an accurate result. This problem can be solved by recent technological advancements such as Mass spectrometry. Secondly, although most analytes are stable on DBS, some unstable compounds are quite challenging in storage due to their interaction with enzyme inhibitors. The presence of blood cells in blood diagnosis may also interfere with the diagnostic quantification, resulting in low sensitivity, unreliable results or false negative. Hematocrit level, measured by the ratio of the volume occupied by packed red blood cells to the volume of the whole blood is also a variable factor that affects the performance of DBS cards. Hence, separation of plasma from the whole blood is preferred to avoid the interference of blood cells, promoting the development of dry plasma spot (DPS) cards, a popular alternation of DBS cards.

Instead of taking the whole blood, DPS targets on the plasma. In the composition of the whole blood, plasma contributes to almost 55% of the blood volume. Plasma mainly consists of water; however, the rest compounds, for instance, blood proteins, nutrients, hormones etc., are crucial to human beings as they could be used for clinical diagnosis. Cell-free plasma is always preferred in clinics. Many standards of care are based on extracted plasma through centrifugation. However, centrifugation may not be desired in the DBS application, where samples are normally collected on site at a small volume. In addition, there is a lack of DPS device that is able to separate blood cells and absorb the plasma simultaneously.

Accordingly, there is a need for a DPS device that ameliorates one or more disadvantages mentioned above. There is a need to provide a membrane for use in such a DPS device that ameliorates one or more disadvantages mentioned above. There is a need to provide a method of forming such a membrane that ameliorates one or more disadvantages mentioned above.

### SUMMARY

In one aspect, there is provided a multi-layered membrane for separating components in an aqueous sample comprising: a porous layer for separating or retaining at least one component from said aqueous sample therein; and an absorbent layer comprising a superabsorbent or absorbent material for removing or retaining liquid from said porous layer.

The aqueous sample may be a biological sample such as a whole blood sample or blood plasma sample.

Advantageously, the multi-layered membrane may demonstrate at least 95%, or about 100% retention of blood cells, thus eliminating the presence of blood cells in the separated blood plasma, resulting in higher sensitivity and reliability of blood plasma diagnosis.

Further advantageously, the multi-layered membrane may enhance blood plasma permeability, thus enabling higher plasma recoveries which in turn improve the accuracy of clinical tests.

Still further advantageously, the multi-layered membrane may result in plasma recovery of about 10% to about 40% from total blood volume.

Still further advantageously, the multi-layered membrane may enhance the permeation rate of biomolecules such as amino acids and blood proteins, enabling higher biomolecules recoveries which in turn improve the accuracy of clinical tests.

Still further advantageously, the separated blood plasma is dehydrated (or at least with minimal amount of water) since the absorbent layer absorbs most if not all of the liquid from the sample. The dehydration of the separated blood plasma helps to stabilise and preserve the blood plasma sample.

In another aspect, there is provided a method of preparing a multi-layered membrane comprising a porous layer and an absorbent layer, the method comprising the steps of:

(a) providing a dope solution of a porous layer material in a solvent;

(b) casting the dope solution to form the porous layer via a method selected from the group consisting of electrospinning, non-solvent induced phase separation (NIPS), thermally induced phase separation (TIPS), vapor induced phase separation (VIPS), a combination of NIPS and TIPS (N-TIPS), and combinations thereof; and

(c) incorporating the absorbent layer adjacent to the porous layer via physical interaction or chemical treatment, wherein the absorbent layer comprises a superabsorbent or absorbent material for removing or retaining liquid from said porous layer.

In another aspect, there is provided a method of separating blood plasma from a whole blood sample, comprising applying said whole blood sample to a multi-layered membrane, wherein said multi-layered membrane comprises a porous layer and an absorbent layer comprising a superabsorbent or absorbent material for removing or retaining liquid from said porous layer.

Advantageously, the method may allow for simultaneous separation of blood plasma from whole blood and dehydration of the separated blood plasma. This simultaneous method may offer a simple method of sample collection, storage and transfer, with reduced infection risk of pathogens.

Further advantageously, the method may be a simple and cheap method for patients to collect their own bio-samples at the convenience of their homes and send the bio-samples for central analysis at national hospitals.

In another aspect, there is provided a diagnostic device for separation of blood plasma from a whole blood sample, comprising a multi-layered membrane comprising a porous layer and an absorbent layer comprising a superabsorbent or absorbent material for removing liquid from said porous layer.

Definitions

The following words and terms used herein shall have the meaning indicated:

The term “multi-layered” is to be interpreted broadly to include bilayered, trilayered, and the like.

The term “porous” as used herein means having pores with an effective pore diameter in the range of 0.1 μm to more than 30 μm and a pore density in the range of 40% to 95%.

The term “plasma recovery” refers to the percentage of plasma in a blood sample that has permeated through the porous layer to land on the absorbent layer. When the absorbent layer is a piece of filter paper, the formula for the calculation of plasma recovery is Plasma Recovery (%)=(weight of filter paper after adsorption−weight of filter paper before adsorption)/(density of plasma×total feed blood volume).

The term “top surface” refers to the surface of a membrane that is facing up when formed comprising a porous structure as described herein.

The term “bottom surface” refers to the surface of a membrane that is facing down when formed comprising a porous structure as described herein. In an asymmetric membrane, the “bottom surface” is to be distinguished from the “top surface” by having larger pores as compared to the “top surface”, where the pore sizes of the larger pores may be greater than 30 μm.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of a multi-layered membrane for separating components in an aqueous sample will now be disclosed.

The multi-layered membrane comprises a porous layer for separating or retaining at least one component from said aqueous sample therein; and an absorbent layer comprising a superabsorbent or absorbent material for removing liquid from said porous layer.

In the multi-layered membrane, the porous layer may contain pores having, in general, an effective pore diameter in the range of about 0.1 μm to more than about 30 μm, about 0.1 μm to about 25 μm, about 0.1 μm to about 10 μm, about 0.1 μm to about 5 μm, about 0.1 μm to about 2 μm, about 0.1 μm to about 1 μm, about 1 μm to about 3 μm, about 2 μm to about 3 μm, about 0.25 μm to about 3 μm, about 0.1 μm to about 3 μm, about 1 μm to more than about 30 μm, about 3 μm to more than about 30 μm, about 5 μm to more than about 30 μm, about 10 μm to more than about 30 μm, about 15 μm to more than about 30 μm, about 20 μm to more than about 30 μm, or about 25 μm to more than about 30 μm.

In the multi-layered membrane, the porous layer may have a pore density in the range of about 40% to about 95%, about 40% to about 90%, about 40% to about 80%, about 40% to about 70%, about 40% to about 60%, about 40% to about 50%, about 50% to about 95%, about 60% to about 95%, about 70% to about 95%, about 80% to about 95% or about 90% to about 95%.

The porous layer may comprise a symmetric or an asymmetric membrane matrix.

The porous layer comprising a symmetric membrane matrix may have the same range of pore sizes and pore densities on all its surfaces and within the porous layer itself, such as those defined above. The peelable matrix layer comprising an asymmetric membrane matrix may have different ranges of pore sizes and pore densities on different surfaces. Therefore, the pore size of the pores at the bottom surface of the porous layer may be greater than 30 μm, while the pore size of the pores at the top surface of the porous layer may be in the range of about 0.1 μm to about 3 μm (including sub-ranges and discrete values therein). Within the porous layer itself, the pore sizes may range from values that form a gradient when viewed from the top surface to the bottom surface, therefore, the pore sizes of the pores within the porous layer can range from about 0.1 μm to more than about 30 μm (including sub-ranges and discrete values therein) depending on whether they are nearer to the top surface or nearer to the bottom surface. The pores within the porous layer may be continuous from the top surface to the bottom surface, or may be dis-continuous and instead form pockets within the porous layer.

In the multi-layered membrane, the porous layer may be further modified to prevent blood clotting and reduce free radicals. Such modifications may include coating, surface modifying or adding anti-coagulant agents or polymers, as appropriate, to the porous layer, which would be within the knowledge of a person skilled in the art.

The porous layer may be a hydrophilic porous layer, a hydrophobic porous layer or combinations thereof.

In the multi-layered membrane, the material of the porous layer is not particularly limited and exemplary materials may be polyacrylonitrile (PAN), polyethersulfone (PES), cellulose acetate (CA), sulfonated polysulfone (SPSf), sulfonated polyethersulfone (SPES), cellulose acetate butyrate, ethylcellulose, hydroxylpropyl cellulose, polyurethane, poloxamer polyols, poly(vinyl alcohol), poly(vinyl chlorine), polytetrafluoroethylene (PTFE), polyvinylidene difluoride (PVDF) or combinations thereof.

The separating or retaining step comprises adding a drop of the aqueous sample on the multi-layered membrane. The aqueous sample then flows through the pores in the multi-layered membrane by gravity. Components larger than the pores of the porous layer are thus retained on the surface or inside the porous layer and become separated from components smaller than the pores of the porous layer that can flow through the pores and become absorbed by the absorbent layer.

The aqueous sample is not particularly limited and exemplary samples may be blood, plasma, urine, saliva or combinations thereof.

The separating or retaining step may separate or retain analytes in the aqueous sample from impurities. The analytes may be analysed for detection of disease.

The aqueous sample may comprise components larger than the pores of the porous layer and components smaller than the pores of the porous layer.

The components larger than the pores of the porous layer are not particularly limited and exemplary components may be red blood cells, white blood cells, platelets or combinations thereof.

The components smaller than the pores of the porous layer are not particularly limited and exemplary components may be small molecules, antigens, antibodies, DNAs, proteins, or combinations thereof.

In the multi-layered membrane, the porous layer may be placed above the absorbent layer.

The porous layer and the absorbent layer may be in physical contact with each other.

The porous layer and the absorbent layer may be held in place by gravity, glue, tapes, staples, magnetic force, heat press, hydraulic press, using of self-adhesive cover or combinations thereof.

The porous layer may have a thickness in the range of about 0.5 μm to about 500 μm, about 5 μm to about 500 μm, about 50 μm to about 500 μm, about 0.5 μm to about 50 μm or about 0.5 μm to about 5 μm.

The porous layer may have an area in the range of about 1 cm² to about 10000 cm², about 10 cm² to about 10000 cm², about 100 cm² to about 10000 cm², about 1000 cm² to about 10000 cm², about 1 cm² to about 1000 cm², about 1 cm² to about 100 cm² or about 1 cm² to about 10 cm².

The absorbent layer may have a thickness in the range of about 10 μm to about 1000 μm, about 100 μm to about 1000 μm, about 500 μm to about 1000 μm, about 10 μm to about 500 μm or about 10 μm to about 100 μm.

The absorbent layer may have an area in the range of about 0.05 cm² to about 100 cm², about 1 cm² to about 100 cm², about 10 cm² to about 100 cm², about 50 cm² to about 100 cm², about 0.05 cm² to about 1 cm², about 0.05 cm² to about 10 cm² or about 0.05 cm² to about 5 cm².The absorbent layer may have an area which is at least the same as the porous layer to absorb all components from the aqueous sample that has flowed through the porous layer.

In the multi-layered membrane, the superabsorbent or absorbent material used is not particularly limited and exemplary materials may be sodium polyacrylate, polyacrylic acid, alginic acid, starch, hydroxylethyl starch, modified starch, alpha cellulose, modified cellulose, chitosan, carboxylmethyl cellulose, montmorillonite, polyvinyl alcohol, polyethylene oxide, polyacrylamide, hydrolysed polyacrylonitrile, dextran, carboxylmethyl dextran, carbon nanotubes, silica, cotton, rayon, cellulosic pulp, synthetic pulp, bamboo silk, zeolite, glass fibers, polyester fibers, polyethylene fibers, fleece, and mixtures thereof.

The multi-layered membrane may further comprise a top layer comprising a peelable matrix layer.

The peelable matrix layer and the porous layer may be in physical contact with each other

The peelable matrix layer and the porous layer may be held in place by gravity, glue, tapes, staples, magnetic force, heat press, hydraulic press, self-adhesive cover or combinations thereof.

The peelable matrix layer may retain components from the aqueous sample which are larger than its pores. The peelable matrix layer with retained components may be peeled off for analysis of the components.

The peelable matrix layer may contain pores having, in general, an effective pore diameter in the range of about 0.1 μm to more than about 30 μm, about 0.1 μm to about 25 μm, about 0.1 μm to about 10 μm, about 0.1 μm to about 5 μm, about 0.1 μm to about 2 μm, about 0.1 μm to about 1 μm, about 1 μm to about 3 μm, about 2 μm to about 3 μm, about 0.25 μm to about 3 μm, about 0.1 μm to about 3 μm, about 1 μm to more than about 30 μm, about 3 μm to more than about 30 μm, about 5 μm to more than about 30 μm, about 10 μm to more than about 30 μm, about 15 μm to more than about 30 μm, about 20 μm to more than about 30 μm, or about 25 μm to more than about 30 μm.

The peelable matrix layer may have a pore density in the range of about 40% to about 95%, about 40% to about 90%, about 40% to about 80%, about 40% to about 70%, about 40% to about 60%, about 40% to about 50%, about 50% to about 95%, about 60% to about 95%, about 70% to about 95%, about 80% to about 95% or about 90% to about 95%.

The peelable matrix layer may comprise a symmetric or an asymmetric membrane matrix.

The peelable matrix layer comprising a symmetric membrane matrix may have the same range of pore sizes and pore densities on all its surfaces. The peelable matrix layer comprising an asymmetric membrane matrix may have different ranges of pore sizes and pore densities on different surfaces. Therefore, the pore size of the pores at the bottom surface of the peelable matrix layer may be greater than 30 μm, while the pore size of the pores at the top surface of the peelable matrix layer may be in the range of about 0.1 μm to about 3 μm (including sub-ranges and discrete values therein). Within the peelable matrix layer itself, the pore sizes may range from values that form a gradient when viewed from the top surface to the bottom surface, therefore, the pore sizes of the pores within the peelable matrix layer can range from about 0.1 μm to more than about 30 μm (including sub-ranges and discrete values therein) depending on whether they are nearer to the top surface or nearer to the bottom surface. The pores within the peelable matrix layer may be continuous from the top surface to the bottom surface, or may be dis-continuous and instead form pockets within the peelable matrix layer.

The peelable matrix layer may be a hydrophilic peelable matrix layer, a hydrophobic peelable matrix layer or combinations thereof.

In the multi-layered membrane, the material of the peelable matrix layer is not particularly limited and exemplary materials may be polyacrylonitrile (PAN), polyethersulfone (PES), cellulose acetate (CA), sulfonated polysulfone (SPSf), sulfonated polyethersulfone (SPES), cellulose acetate butyrate, ethylcellulose, hydroxylpropyl cellulose, polyurethane, poloxamer polyols, poly(vinyl alcohol), poly(vinyl chlorine), polytetrafluoroethylene (PTFE), polyvinylidene difluoride (PVDF) or combinations thereof.

The peelable matrix layer may have a thickness in the range of about 0.5 μm to about 500 μm, about 5 μm to about 500 μm, about 50 μm to about 500 μm, about 0.5 μm to about 50 μm or about 0.5 μm to about 5 μm.

The peelable matrix layer may have an area in the range of about 0.1 cm² to about 100 cm², about 10 cm² to about 50 cm², about 50 cm² to about 100 cm², about 0.1 cm² to about 1 cm², about 1 cm² to about 100 cm², about 1 cm² to about 10 cm² or about 0.1 cm² to about 1 cm².The peelable matrix layer may have an area which is at most the same as the porous layer so all the components of the aqueous sample that has flowed through the peelable matrix layer may enter the porous layer.

Exemplary, non-limiting embodiments of a method of preparing a multi-layered membrane comprising a porous layer and an absorbent layer will now be disclosed.

The method comprises the steps of (a) providing a dope solution of a porous layer material in a solvent; (b) casting the dope solution to form the porous layer via a method selected from the group consisting of electrospinning, non-solvent induced phase separation (NIPS), thermally induced phase separation (TIPS), vapor induced phase separation (VIPS), a combination of NIPS and TIPS (N-TIPS), and combinations thereof; and (c) incorporating the absorbent layer adjacent to the porous layer via physical interaction or chemical treatment, wherein the absorbent layer comprises a superabsorbent or absorbent material for removing liquid from said porous layer. The method may be used to prepare the multi-layered membrane as described above, wherein the multi-layered membrane comprises a porous layer and an absorbent layer comprising a superabsorbent or absorbent material for removing liquid from said porous layer.

The step (a) may be undertaken with the dope solution having a concentration of porous layer material in the range of about 3.0 weight % to about 10.0 weight %, about 3.0 weight % to about 9.0 weight %, about 3.0 weight % to about 7.0 weight %, about 3.0 weight % to about 5.0 weight %, about 5.0 weight % to about 10.0 weight %, about 7.0 weight % to about 10.0 weight % or about 9.0 weight % to about 10.0 weight %.

In step (a), the solventused is not particularly limited and exemplary solvents may be N-methylpyrrolidone (NMP), dimethylformamide (DMF), dimethylacetamide (DMAc), dimethyl sulfoxide (DMSO), hexafluoroisopropanol and combinations thereof.

The dope solution in step (a) may further comprise additives. The dope solution in step (a) may be a combination of the solvent and the additive.

The additives used are not particularly limited and exemplary additives may be methanol, ethanol, isopropanol, acetone, tetrahydrofuran, water, glycerol, ethylene glycol and combinations thereof.

The additives can be used to tune the pore size, porosity and structure of formed membranes.

The step (a) may be undertaken with the dope solution having a concentration of combined solvent and additives in the range of about 90.0 weight % to about 97.0 weight %, about 90.0 weight % to about 95.0 weight %, about 90.0 weight % to about 93.0 weight %, about 90.0 weight % to about 91.0 weight %, about 91.0 weight % to about 97.0 weight %, about 93.0 weight % to about 97.0 weight % or about 95.0 weight % to about 97.0 weight %.

In step (a), the porous layer material used may be a hydrophilic material, a hydrophobic material or combinations thereof.

In step (a), the porous layer material used is not particularly limited and exemplary materials may be polyacrylonitrile (PAN), polyethersulfone (PES), sulfonated polysulfone (SPSf), sulfonated polyethersulfone (SPES), cellulose acetate (CA), cellulose acetate butyrate, ethylcellulose, hydroxylpropyl cellulose, polyurethane, poloxamer polyols, poly(vinyl alcohol), poly(vinyl chlorine), polytetrafluoroethylene (PTFE), polyvinylidene difluoride (PVDF) and combinations thereof.

In step (a), the dope solution is provided by mixing the porous layer material with the solvent, optionally with the additives.

In one embodiment, the mixing step may be performed by adding the porous layer material into the solvent, and stirring this for a period of time and temperature. The stirring speed may be in the range of about 50 rpm to about 150 rpm, at a temperature in the range of about 50° C. to about 100° C. and for a period of time of about 6 hours to about 18 hours. The dope solution may then be cooled down to room temperature for addition of the additives. The dope solution may further be stirred at room temperature until a homogeneous solution is achieved, where the stirring speed may be in the range of about 50 rpm to about 150 rpm.

In one embodiment, the mixing step may be performed by adding the porous layer material into a mixture of the solvent and the additives. The dope solution may then be stirred for a period of time and temperature until a homogeneous solution is achieved. The stirring speed may be in the range of about 50 rpm to about 150 rpm, at a temperature in the range of about 50° C. to about 100° C. and for a period of time of about 2 hours to about 6 hours. The dope solution made by this method may be used for N-TIPS.

The casting step (b) may be undertaken via the electrospinning method.

In the electrospinning method, the dope solution is loaded into a syringe and then pushed out from a needle at a certain flow rate. By adding a high voltage at the needle's tip, the dope solution that is pushed out can be stretched when electrostatic repulsion from the high voltage overcomes the surface tension of the dope solution, resulting in the formation of nanofibers. The nanofibers can be made into a membrane by collecting them after a prolonged fiber collection time.

The collecting step may be performed using a grounded collector. The collector used is not particularly limited and exemplary collectors may be drum rollers, metallic plates, parallel electrodes or combinations thereof.

The collecting step may be performed using a drum roller at a roller speed. The roller speed may be adjusted to modify the physical characteristics of the nanofibers collected.

The casting step (b) may be undertaken via the electrospinning method with the fiber collection time in the range of about 15 minutes to about 120 minutes, about 15 minutes to about 90 minutes, about 15 minutes to about 60 minutes, about 15 minutes to about 30 minutes, about 30 minutes to about 120 minutes, about 60 minutes to about 120 minutes or about 90 minutes to about 120 minutes. The collection time may be about 30 minutes.

The casting step (b) may be undertaken via the electrospinning method with the roller speed in the range of about 70 rpm to about 1000 rpm, about 70 rpm to about 800 rpm, about 70 rpm to about 600 rpm, about 70 rpm to about 400 rpm, about 70 rpm to about 200 rpm, about 200 rpm to about 1000 rpm, about 400 rpm to about 1000 rpm, about 600 rpm to about 1000 rpm or about 800 rpm to about 1000 rpm.

The casting step (b) may be undertaken via the NIPS, TIPS, VIPS or N-TIPS method.

In the NIPS, TIPS, VIPS and N-TIPS methods, the dope solution may be poured on a casting plate at high temperature and then spread out across the casting plate using a casting knife. The dope solution that is spread out may be converted to a solid membrane subsequently by additive treatment, cooling treatment, vapor treatment or a combination of additive treatment and cooling treatment for NIPS, TIPS, VIPS or N-TIPS, respectively.

The casting plate is not particularly limited and exemplary casting plates may be a glass, a belt, a metal or combinations thereof.

In the spreading step, the casting knife may be kept at a height above the casting plate. The height of the casting knife may be adjusted to modify the thickness of the solid membrane formed.

The casting step (b) may be undertaken via the NIPS, TIPS or N-TIPS method with the height of the casting knife in the range of about 50 μm to about 500 μm, about 50 μm to about 400 μm, about 50 μm to about 300 μm, about 50 μm to about 200 μm, about 50 μm to about 100 μm, about 100 μm to about 500 μm, about 200 μm to about 500 μm, about 300 μm to about 500 μm or about 400 μm to about 500 μm.

The casting step (b) may be undertaken via the electrospinning method with a weight percent ratio of the solvent and additives is in the range of about 100:1 to about 3:1, about 100:1 to about 9:1, about 100:1 to about 20:1, about 100:1 to about 50:1, about 50:1 to about 3:1, about 20:1 to about 3:1 or about 9:1 to about 3:1.

The casting step (b) may be undertaken via the TIPS method with a partial dope phase separation through VIPS process.

The casting step (b) may be undertaken via the TIPS method with the porous layer material being PAN, the solvent being a mixed solvent of dimethyl sulfoxide DMSO/water at 85/15% by volume.

In the casting step (b), the concentration of the porous layer material may be in the range of about 40.0 mg/ml to about 120.0 mg/ml, about 40.0 mg/ml to about 100.0 mg/ml, about 40.0 mg/ml to about 80.0 mg/ml, about 40.0 mg/ml to about 60.0 mg/ml, about 60.0 mg/ml to about 120.0 mg/ml, about 80.0 mg/ml to about 120.0 mg/ml, about 100.0 mg/ml to about 120.0 mg/ml, about 60.0 mg/ml to about 70.0 mg/ml or about 60.0 mg/ml to about 65.0 mg/ml.

The casting step (b) may be undertaken via the N-TIPS method with the cooling treatment and additive treatment of the dope solution being cooling in an additive at 25° C. The additive may optionally be provided as a mixture with a solvent. The solvent used is not particularly limited and exemplary solvents may be N-methylpyrrolidone (NMP), dimethylformamide (DMF), dimethylacetamide (DMAc), dimethyl sulfoxide (DMSO), hexafluoroisopropanol and combinations thereof.

The mixture may have a volume ratio of the additive and the solvent in the range of about 1:9 to about 5:5, about 2:8 to about 5:5, about 3:7 to about 5:5, about 4:6 to about 5:5, about 1:9 to about 4:6, about 1:9 to about 3:7 or about 1:9 to about 2:8.

The casting step (b) may be undertaken via the N-TIPS method with the porous layer material being PAN.

The concentration of the porous layer material may be in the range of about 3.60 weight % to about 6.50 weight %, about 3.60 weight % to about 6.00 weight %, about 3.60 weight % to about 5.00 weight %, about 3.60 weight % to about 4.00 weight %, about 4.00 weight % to about 6.50 weight %, about 5.00 weight % to about 6.50 weight % or about 6.00 weight % to about 6.50 weight % of the dope solution.

The porous layer may be further modified physically or chemically to contain specific binding sites for desired molecules.

In step (c), the physical interaction to hold the absorbent layer and the porous layer adjacent to each other may comprise gravity, tapes, staples, magnetic force, heat press, hydraulic press, self-adhesive cover or combinations thereof.

In step (c), the chemical interaction to hold the absorbent layer and the porous layer adjacent to each other may comprise forming cross-linked polymers, forming hydrogen bonding or combinations thereof.

Exemplary, non-limiting embodiments of a method of separating blood plasma from a whole blood sample will now be disclosed. The method comprises applying the whole blood sample to a multi-layered membrane.

The multi-layered membrane may comprise a porous layer and an absorbent layer. The absorbent layer may comprise a superabsorbent or absorbent material for removing liquid from the porous layer.

In the applying step, the components of the whole blood sample larger than the pores of the porous layer may be retained above or within the porous layer. The components of the whole blood sample smaller than the pores of the porous layer may flow through the pores and subsequently be absorbed by the absorbent layer.

In the method, the whole blood sample may be applied to the bottom surface of the porous layer where there are larger pores of greater than 30 μm pore size as compared to the top surface of the porous layer.

When the whole blood sample is applied to the bottom surface, the contact between the top surface and the absorbent layer may provide an additional capillary force to improve the flow of the whole blood sample through the membrane.

When the whole blood sample is applied to the bottom surface, the whole blood sample may have an improved spreading due to larger pore sizes of the bottom surface, thus increasing improving the flow of the whole blood sample through the membrane.

The multi-layered membrane may be as defined above.

Exemplary, non-limiting embodiments of a diagnostic device for separation of blood plasma from a whole blood sample will now be disclosed. The diagnostic device comprises a multi-layered membrane.

The multi-layered membrane may comprise a porous layer and an absorbent layer. The absorbent layer may comprise a superabsorbent or absorbent material for removing liquid from the porous layer. The multi-layered membrane may be as defined above.

The diagnostic device may further comprise a blood filter above the multi-layered membrane.

The blood filter may remove clots and small clumps of platelets which are formed when the whole blood sample is taken. This may leave the whole blood sample with isolated blood cells and smaller components, thus improving the diagnostic device's accuracy and lifespan.

The blood filter may comprise a porous membrane of a biocompatible polymer.

The porous membrane may have pores with a mean effective diameter in the range of about 10 μm to about 300 μm, about 100 μm to about 300 μm, about 200 μm to about 300 μm, about 10 μm to about 200 μm or about 10 μm to about 100 μm.

The porous membrane may have a thickness in the range of about 0.5 mm to about 2 mm, about 1 mm to about 2 mm, about 1.5 mm to about 2 mm, about 0.5 mm to about 1.5 mm or about 0.5 mm to about 1 mm.

The biocompatible polymer is not particularly limited and exemplary biocompatible polymers may be polyester, polycarbonate, polyacrylamide or combinations thereof.

When the diagnostic device is used to separate and dehydrate the plasma from blood cells (as shown in FIG. 1) a sample of blood is dropped onto the device surface, blood cells may be retained and prevented from entering the multi-layered membrane due to size exclusion. Driven by gravity, the liquid plasma will penetrate into the multi-layered membrane together with components smaller than the pores of the porous layer. To prevent samples from degradation, an absorbent layer on the bottom is specially incorporated to completely dehydrate the plasma.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a schematic illustration of a diagnostic device 100 for plasma separation and dehydration from blood cells. The diagnostic device 100 comprises a multi-layered membrane 3. The diagnostic device may further comprise a blood filter 2. The multi-layered membrane 3 (as expanded from the circular region) comprises a porous layer 5 and an absorbent layer 6. A sample of blood 1 may be separated by the device into retained blood cells 4 and plasma which is absorbed into the absorbent layer 6.

FIG. 2 is a schematic diagram of a method used to assess the performance of the formed multi-layered membrane 3. Within a sample of blood 1, only plasma 7 may permeate through the porous layer 5 to be absorbed by the absorbent layer 6. The top surface 8 and the bottom surface 9 are marked in FIG. 2.

FIG. 3A to FIG. 3O are images showing the influence of fiber collection time on morphology, the appearance of blood drops, plasma recovery and red blood cell retention of formed porous layer. FIG. 3A to FIG. 3E are field emission scanning electron microscope (FESEM) images of the membranes; FIG. 3F to FIG. 3J are photographic images of the top surface 8 of the membrane where a sample of blood is applied; FIG. 3K to FIG. 3O are photographic images of the absorbent layer 6. The fiber collection time is 15 minutes for FIGS. 3A, 3f, 3K; 30 minutes for FIGS. 3B, 3G, 3L; 60 minutes for FIGS. 3C, 3H, 3M; 90 minutes for FIGS. 3D, 3I, 3N or 120 minutes for FIGS. 3E, 3J, 3O. The plasma recovery is 11.45±0.47% for FIGS. 3A, 3F, 3K; 10.30±0.53% for FIGS. 3B, 3G, 3L; 10.71±3.05% for FIGS. 3C, 3H, 3M; 2.69±0.51% for FIGS. 3D, 3I, 3N or 0.94±0.23% for FIGS. 3E, 3J, 3O.

FIG. 4A to FIG. 4X are images showing the influence of solvent and solvent/additive ratio on plasma recoveries and membrane morphologies of the porous layer formed by the electrospinning method, where FIGS. 4Ato 4L show FESEM images of the porous layer's morphology and FIGS. 4M to 4X show camera images of the absorbent layer. The solvent used is N-methylpyrrolidone (NMP) for FIGS. 4A to 4D and 4M to 4P; Dimethylformamide (DMF) for FIGS. 4E to 4H and 4Q to 4T or (c) Dimethylacetamide (DMAc) for FIGS. 4I to 4L and 4U to 4X. Using acetone as the additive, the solvent/additive ratio is 100/0 for FIGS. 4A, 4E, 4I, 4M, 4Q, 4U; 19/1 for FIGS. 4B, 4F, 4J, 4N, 4R, 4V; 9/1 for FIGS. 4C, 4G, 4K, 4O, 4S, 4W or 8/2 for FIGS. 4D, 4H, 4L, 4P, 4T, 4X. The plasma recovery is 6.67±0.31% for FIGS. 4A and 4M; 9.96±0.84% for FIGS. 4E and 4Q; 6.94±0.31% for FIGS. 4I and 4U; 7.27±0.81% for FIGS. 4B and 4N; 11.64±1.11% for FIGS. 4F and 4R; 5.32±1.11% for FIGS. 4J and 4V; 9.63±1.22% for FIGS. 4C and 4O; 21.54±2.68% for FIGS. 4G and 4S; 13.33±1.21% for FIGS. 4K and 4W 10.30±0.53% for FIGS. 4D and 4P; 21.07±0.31% for FIGS. 4H and 4T or 27.94±1.76% for FIGS. 4L and 4X. Red blood cells are observed on FIGS. 4S, 4T and 4X.

FIG. 5A to FIG. 5I are FESEM images showing the influence of polymer concentration on morphologies of the top surface (FIGS. 5A, 5D and 5G), the bottom surface (FIGS. 5B, 5E and 5H) and the vertical cross-section (FIGS. 5C, 5F and 5I) of the porous layer made from thermally induced phase separation (TIPS) with 87.0 mg/ml (FIGS. 5A to 5C), 63.8 mg/ml (FIGS. 5D to 5F) or 41.7 mg/ml (FIGS. 5G to 5I) polyacrylonitrile (PAN).

FIG. 6A to FIG. 6I are camera images showing the influence of polymer concentration on the plasma recovery and the red blood cell retention of the top surface (FIGS. 6A, 6D and 6G) and the bottom surface (FIGS. 6B, 6E and 6H) of the porous layer made from TIPS with 87.0 mg/ml (FIGS. 6A to 6C), 63.8 mg/ml (FIGS. 6D to 6F) or 41.7 mg/ml PAN (FIGS. 6G to 6J). Images of the absorbent layer are provided in FIGS. 6C, 6F and 6I. The membrane formed by the condition of as shown in FIGS. 6A to 6C had a plasma recovery of 1.81% while red blood cells have flowed through the membranes formed by the conditions as shown in FIGS. 6D to 6I.

FIG. 7A to FIG. 7I are a series of FESEM images of the top surface (FIGS. 7A, 7D and 7G), the bottom surface (FIGS. 7B, 7E and 7H) and the vertical cross-section (FIGS. 7C, 7F and 7I) of the porous layer made from TIPS with 87.0 mg/ml PAN. The membranes were cooled in air for 1 hour on a hot plate that is cooled from 90° C. (FIGS. 7A to 7C), in room temperature (FIGS. 7D to 7F) or in water (FIGS. 7G to 7J). The membrane formation further included a step of additive induced phase separation (NIPS) when cooled in water, being made by N-TIPS.

FIG. 8A to FIG. 8L are a series of camera images showing the influence of cooling methods on the plasma recovery and the red blood cell retention of the top surface (FIGS. 8A, 8D, 8G and 8J) and the bottom surface (FIGS. 8B, 8E, 8H and 8K) of the porous layer cooled on a hot plate that is cooled from 90° C. (FIGS. 8A to 8C), in room temperature (FIGS. 8D to 8E), and in water (FIGS. 8F to 8L). Images of the absorbent layer are provided in FIGS. 8C, 8F, 8I and 8L. In FIGS. 8A to 8I, the sample of blood was applied on the top surface 8 of the porous layer, while in FIGS. 8J to 8L, the sample of blood was applied on the bottom surface 9 of the porous layer, which was flipped vertically before use. The plasma recovery is 1.61% for FIGS. 8A to 8C, 1.81% for FIGS. 8D to 8F, 2.83% for FIGS. 8G to 8I or 10.84% for FIGS. 8J to 8L.

FIG. 9A to FIG. 9I are a series of camera images shows the influence of polymer concentration on the plasma recovery and the red blood cell retention of the top surface (FIGS. 9A, 9D and 9G) and the bottom surface (FIGS. 9B, 9E and 9H) of the porous layer made from N-TIPS with 87.0 mg/ml (FIGS. 9A to 9C), 63.8 mg/ml (FIGS. 9D to 9F) or 41.7 mg/ml PAN (FIGS. 9G to 9I). Images of the absorbent layer are provided in FIGS. 9C, 9F and 9Ii. The plasma recovery is 10.84% (FIGS. 9A to 9C) or 33.76% (FIGS. 9D to 9F). Red blood cells have flowed through the membrane of FIGS. 9G to 9I.

FIG. 10A to FIG. 10L are a number of FESEM images on morphologies of the top surface (FIGS. 10A, 10E and 10I), the vertical cross-section (FIGS. 10B, 10F and 10J), the bottom surface (FIGS. 10C, 10G and 10K), and the enlarged bottom surface (FIGS. 10D, 10H and 10L) of the porous layer made from N-TIPS with 87.0 mg/ml PAN. The coagulant used was water (FIGS. 10A to 10D), 70 weight % NMP in water (FIGS. 10E to 10H) or 70 weight % isopropanol (IPA) in water (FIGS. 10I to 10L).

FIG. 11A to FIG. 11F are a series of camera images of the porous layer (FIGS. 11A to 11C) and the absorbent layer (FIGS. 11D to 11F) of the multi-layered membrane after use. The porous layer was prepared with the coagulant of water (FIGS. 11A and 11D), 70 weight % NMP in water (FIGS. 11B and 11E) or 70 weight % IPA in water (FIGS. 11C and 11F).

### EXAMPLES

Non-limiting examples of the invention and a comparative example will be further described in greater detail by reference to specific Examples, which should not be construed as in any way limiting the scope of the invention.

### Example 1

### Structure of the Diagnostic Device

The membrane is of great importance in the DPS devices. A good membrane should have a 100% rejection of blood cells but no retentions to useful analytes. Since suitable membranes for the application are still lacking, the main target would be the development and optimization of required membranes for decellularization via gravity. A few membrane materials, for instance, polyacrylonitrile (PAN), polyethersulfone (PES) and cellulose acetate (CA), were investigated; and different additives were added to the dope solutions to tune the pore sizes and properties of formed membranes.

Furthermore, membranes were formed through a few methods, such as non-solvent induced phase separation (NIPS), electrospinning and thermally induced phase separation (TIPS).

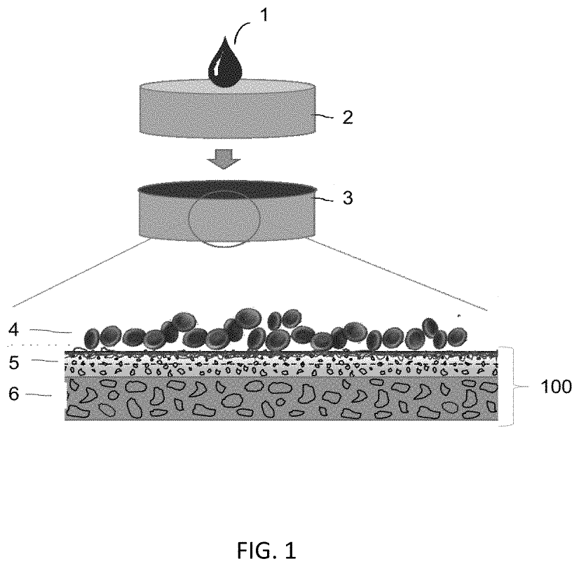

As shown in FIG. 1, a diagnostic device 100 for plasma separation and dehydration from blood cells is provided. The diagnostic device 100 comprises a multi-layered membrane 3. The diagnostic device may further comprise a blood filter 2. The multi-layered membrane 3 (as expanded from the circular region) comprises a porous layer 5 and an absorbent layer 6. A sample of blood 1 may be separated by the device into retained blood cells 4 and plasma which is absorbed into the absorbent layer 6.

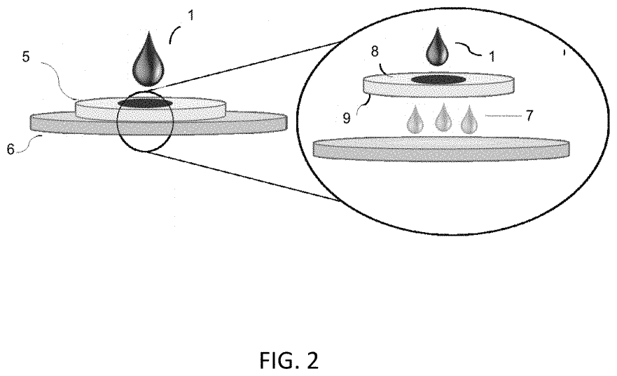

The membranes were then tested with the process shown in FIG. 2. Before testing, the membrane was held together with a filter paper or an absorbent. Blood was then dropped on the top of the membrane. If the plasma could permeate through the membrane and be absorbed by the filter paper, a watermark could be observed on the filter paper. If the watermark turned red, it suggested that red blood cells have passed through the membrane and the membrane was not desired.

The recovery of plasma could be derived from the following formula:

Plasma Recovery (%)=(weight of filter paper after adsorption−weight of filter paper before adsorption)/(density of plasma×total feed blood volume).

Membranes were optimized through two methods, which were electrospinning and TIPS. TIPS may be further combined with NIPS into N-TIPS for the formation of membranes.

### Example 2

### Fabrication Process of the Porous Layer via Electrospinning

The first kind of membranes was formed through the electrospinning process. In electrospinning, the polymer dope solution is pushed out of the syringes filled with the solution at a certain flow rate. By adding a high voltage at the needle tip, the solution droplets coming out of the needle can be stretched when electrostatic repulsion overcomes the surface tension of the solution, resulting in the formation of nanofibers. The nanofiber membrane can be formed by collecting the nanofibrous structures for a prolonged time. The physical properties of the membrane can be tuned by several factors, such as electric potential, dope flow rate, fiber collection time and dope formulas. By choosing proper electrospinning conditions, membranes with optimized performance can be achieved subsequently.

Since PAN has moderate hydrophilicity and has already been applied in the kidney dialysis, it was chosen in this disclosure to form the membrane separator. The polymer (obtained from R&D Center for Membrane Technology, Department of Chemical Engineering, Chung Yuan University, Chung-Li 32023, Taiwan) had a concentration of 9 weight %. N-methylpyrrolidone (NMP, 99.5%, purchased from Merck, Germany) and acetone (Ace, ≥99.8%, AR grade, purchased from Fisher Chemical) were used as the solvent and additives, respectively, at a ratio of 8:2 (weight %) to prepare the polymer solution for electrospinning. The two solvents made up of 91 weight % of the total dope weight. The influence of fiber collection time on membrane performance was investigated first as it determined the thickness and thus the permeability of the formed membranes.

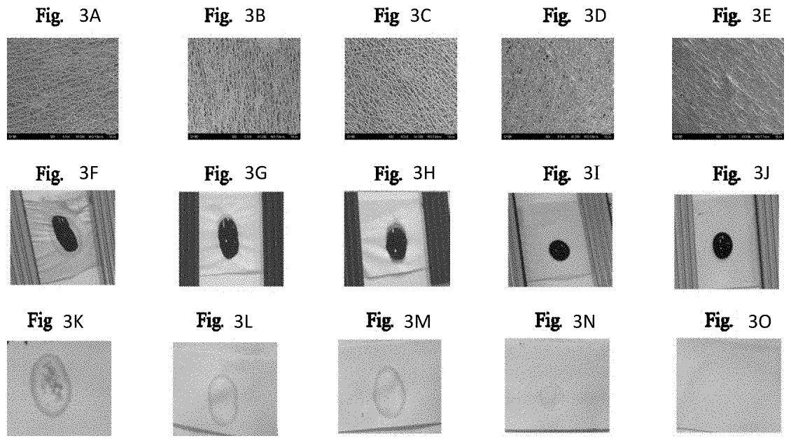

FIGS. 3A to 3O depict the results. At a collection time of 15 minutes, the formed membrane was too thin and porous. Red blood cells could pass through the membrane from defect points and stain the absorbent filter paper. By increasing the collection time, the presence of red blood cells on the filter paper disappeared. However, a decrease in plasma permeation was also observed. The membrane only had a less than 1% plasma recovery when collected for 120 minutes, indicating that a prolonged collection time may generate a membrane too thick to conduct the decellularization application. An optimal collection time could be 30 minutes. A collection time of 30 minutes is choose because (1) the membrane collected by 30 minutes has similar performance as compared to the membrane collected by 60 minutes; (2) the membrane can reject 100% of blood cells; and (3) it saves materials and time during fabrication.

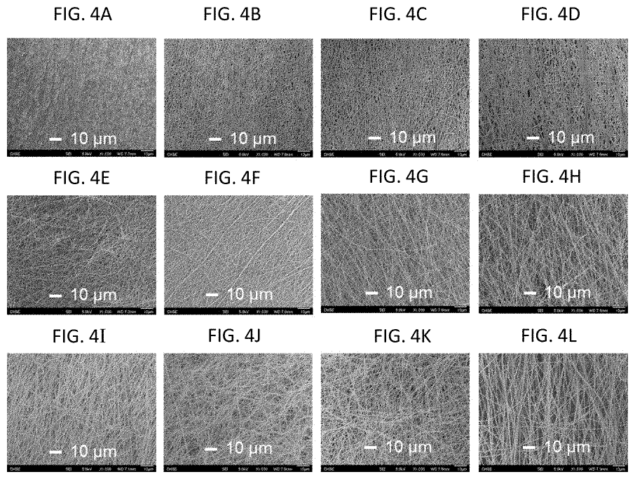

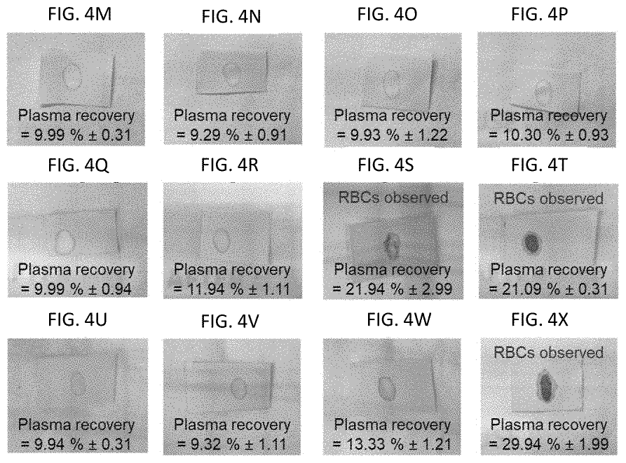

After determining the suitable collection time, the influence of dope formula was subsequently studied, with the results shown in FIGS. 4A to 4X. Since dope solutions contained both solvent and additive, they were manipulated in two ways: (1) substituting NMP to other commonly used solvents such as dimethylformamide (DMF, ≥99.9%, HPLC grade, purchased from VWR Chemicals) or dimethylacetamide (DMAc, ≥99.5%, HPLC grade, purchased from VWR Chemicals) in electrospinning; and (2) varying additive (acetone) to solvent (NMP) ratio. NMP, DMF, and DMAc are good solvents to dissolve PAN. However, they are different in many physical properties, such as boiling point, viscosity, etc. By using different solvents, the viscosities and surface tensions of the polymer solutions could be altered, which in turn affect the evaporation rate of the solvents.

As shown in FIGS. 4A to 4X, membranes made from DMF had a higher plasma recovery as compared to the membranes made from NMP and DMAc. It was easy to interpret as the boiling point of DMF is lower than that of DMAc and NMP. By increasing the acetone to solvent ratio, a higher plasma recovery was discovered. It was due to the formation of a more porous layer from a fast evaporation of acetone. Red blood cells could even pass through the membranes when the membranes were made from DMF or DMAc with high acetone contents. Based on membrane morphologies in FIGS. 4A to 4X, the membranes formed were highly porous and the pore distribution is uniform for the electrospinning membranes, such that the pore size of the formed membranes could be in the range of 0.25 to 3.00 μm.

The best membrane made from electrospinning had a plasma recovery of 13.33±1.21% with almost zero retentions to large molecules, such as human albumin protein (MW: 66.5 kDa). It also had almost 100% permeations of amino acids, for instance, glutamine acid, histidine, etc.

### Example 3

### Fabrication Process of the Porous Layer via Phase Separation

Membranes can also be formed through the thermally induced phase separation (TIPS) process. In this process, polymers are dissolved into a solvent mixture and cast at an elevated temperature. The cast polymer solution will undergo a precipitation process at a lower temperature, resulting in the formation of membranes. Membranes made from TIPS can be tuned in several ways by changing, for instance, dope formula and cooling condition in membrane formation.

The impact of dope formula on membrane morphologies and plasma recoveries were investigated first by varying the polymer concentration in the dope formula. The polymer was dissolved in 100 ml dimethyl sulfoxide (DMSO, 99.9%, ACS reagent, purchased from Sigma-Aldrich)/deionized water (DI water) (85/15 volume %) mixed solvent.

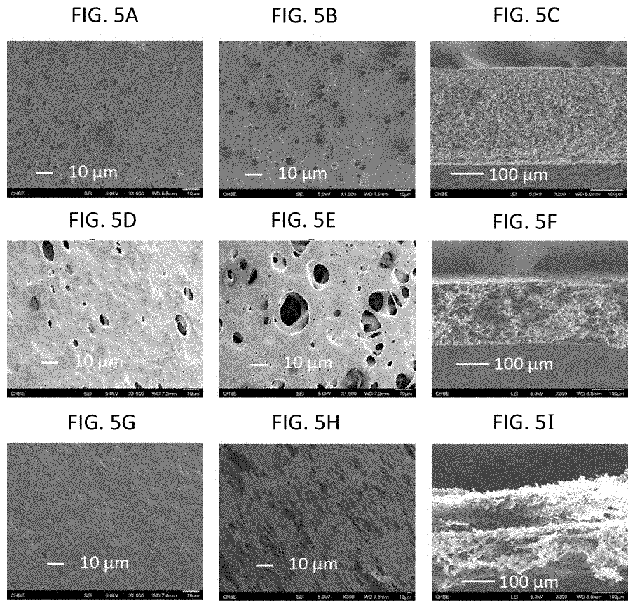

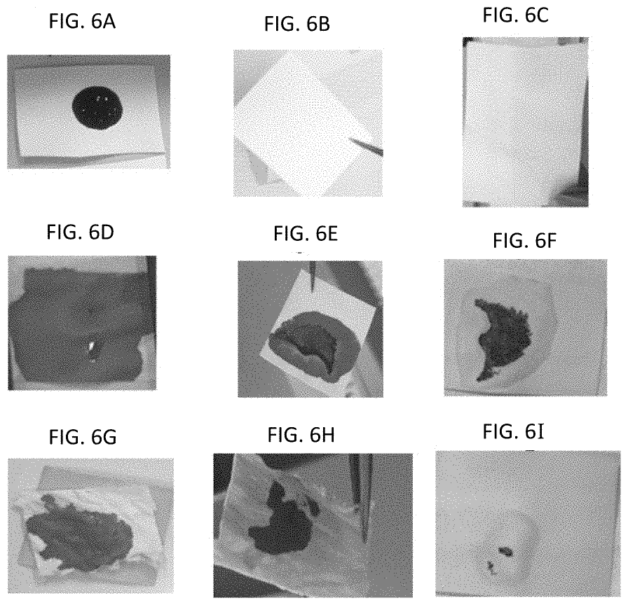

FIGS. 5A to 5I and FIGS. 6A to 6I present the influence of polymer concentration on morphologies and performance of the formed membranes, respectively. By decreasing the polymer concentration from 87.0 mg/ml to 63.8 mg/ml, membranes became more porous with large pores being observed on both top and bottom surfaces of the membrane.

The increased pores of membranes could also be buttressed by the spreading of blood on the membranes in FIGS. 6A to 6I. The blood droplet could spread fast on the membrane made from 63.8 mg/ml PAN, whereas it remained its shape on the membrane made from 87.0 mg/ml PAN. Furthermore, blood could also be observed on the bottom surface of the membrane and the filter paper beneath the membrane for membrane made from 63.8 mg/ml PAN, indicating that the membrane had much larger pore sizes. By further decreasing the polymer concentration of the membranes from 63.8 mg/ml to 41.7 mg/ml, the membrane pore size, however, reduced in the FESEM images. It might be caused by the weak mechanical properties of the membranes made from 41.7 mg/ml PAN, which resulted in shrinkage and loss of morphologies of the membrane during the vacuum drying process. The plasma recovery results reconfirmed that a PAN concentration of 41.7 mg/ml might be too low to form a good membrane in the DPS application because the plasma spot on the filter paper was small and contains red blood cells. Since membranes made from 87.0 mg/ml of PAN had a positive plasma permeation and full retention of red blood cells, the dope formula was used subsequently to investigate the influence of cooling conditions on membrane performance.

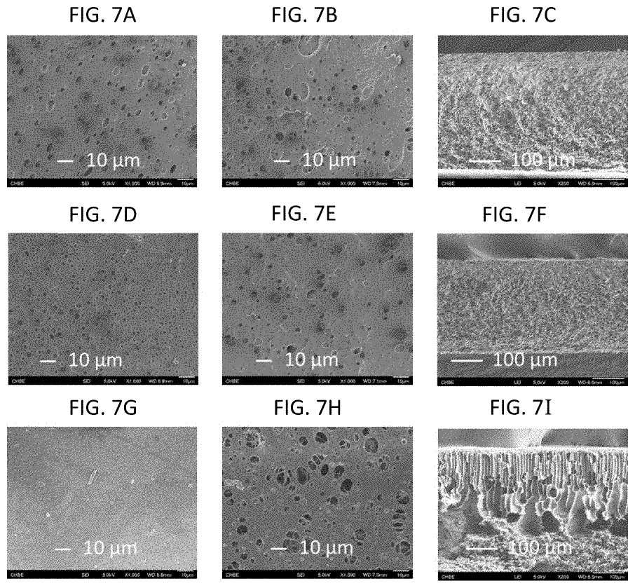

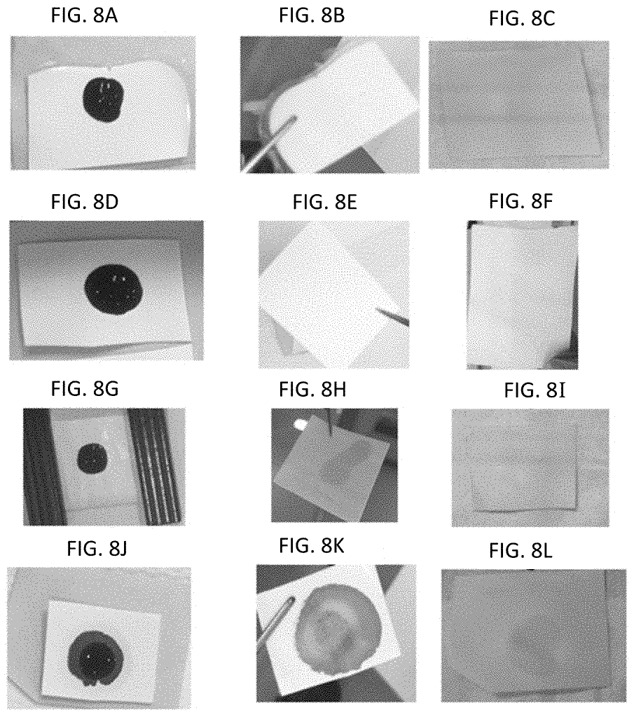

FIGS. 7A to 7I and FIGS. 8A to 8L represent the influence of cooling conditions on the morphologies and the performance of the formed membranes accordingly. Three cooling conditions are chosen; namely: (1) cooling gradually on the hotplate, (2) cooling in room temperature; and (3) cooling in water at room temperature. Compared to the cooling gradually on the hotplate, membranes made from cooling in room temperature might have a slightly more porous structure because they had a small increase in plasma recovery. Even though larger pores were observed on the membrane made from cooling gradually on the hotplate, the membrane could still be relatively dense due to the fast evaporation of the solvents at elevated temperatures. The plasma recovery further increased for the membrane made from cooling in water at room temperature. Comparing to the rest two membranes, the membranes made from cooling in water involved two-phase inversion mechanisms, i.e., TIPS and non-solvent induced phase separation (NIPS). The formed membranes had relatively denser selective layers and more porous bottom surfaces. Their cross-sections also contained finger-like macrovoids, which were caused by the additive (water) intrusion during the NIPS process. The presence of macrovoids might decrease the permeate resistance of plasma, leading to an enhanced plasma recovery. However, a plasma recovery of 2.82% was still low for the membrane to be used in the DPS applications. More effective methods are required to enhance the membrane plasma recoveries.

The selective layer (top surface) of a membrane is the barrier to separate blood cells from blood, especially for asymmetric membranes made from a combination of TIPS and NIPS (N-TIPS). The presence of the support layer (bottom surface) would be a barrier between the selective layer and the absorbent below the membrane, attenuating the function of the absorbent in taking in the plasma. If the membrane is flipped vertically with the supporting layer facing up, the selective layer would be in contact with the filter paper. The contact helps to provide an additional capillary force in addition to the gravity in transportation and separation of blood, facilitating the adsorption of plasma by the absorbent. By flipping the membrane and dropping blood at the membrane bottom surface, a high plasma recovery of 10.84% could be achieved, which was almost 4 times as compared to the one at the original placement. Furthermore, a good spreading of blood could be observed at the membrane's porous bottom surface. It might increase the contact area between the blood spot and the absorbent, which in turn further enhanced the plasma recovery of the membrane.

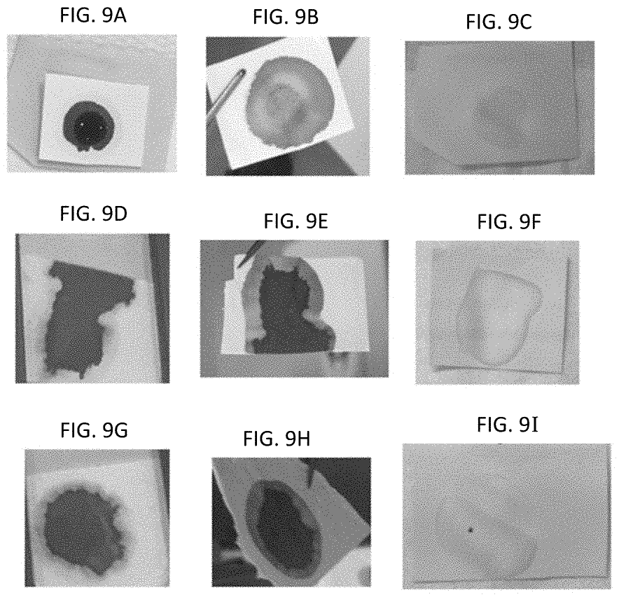

The cooling in water approach was applied to the rest two dope formulas, which were made from 41.7 mg/ml and 63.8 mg/ml PAN, with the results shown in FIGS. 9A to 9I. All fabricated membranes were flipped with blood dropping on the bottom surfaces of the membranes. By decreasing the polymer concentration from 87.0 mg/ml to 63.8 mg/ml, the fabricated membrane had an almost tripled plasma recovery. However, red blood cells started to permeate through the membrane by further decreasing the membrane polymer concentration from 63.8 mg/ml to 41.7 mg/ml. Thus, a polymer concentration of 63.8 mg/ml was the optimal polymer concentration for the membrane formation method of N-TIPS. The membrane made from the method could have a plasma recovery as high as 33.76±4.53%.

Since the membrane made from N-TIPS can have an impressive plasma recovery as high as 33.76±4.53%, it is hypothesized that a better membrane could be formed by changing the coagulant from water to solvent mixtures. By using a coagulant that could induce a slow demixing of the dope solution, a porous layer with large pores could be achieved. Thus, two solvent mixtures, i.e. NMP/water and isopropanol (IPA, 99.5%, purchased from Fisher Chemical)/water, were used in the study.

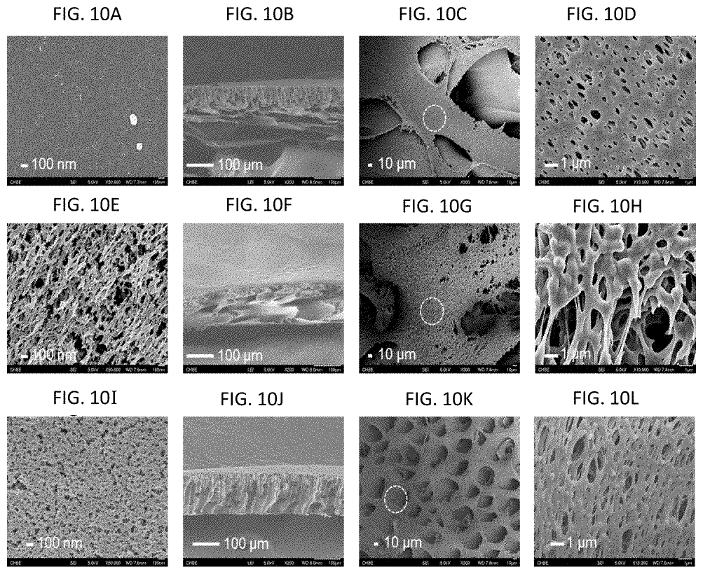

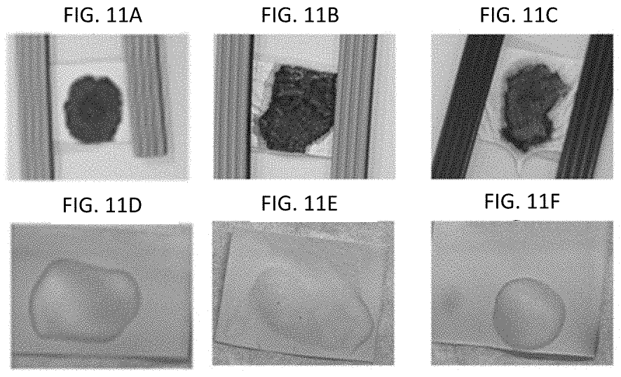

FIGS. 10A to 10L show the morphologies of the membranes made from a combination of TIPS/NIPS by using water, NMP/water or IPA/water as the coagulant. It can be found that the formed membranes had more porous top surfaces with clearly observed pores by using NMP/water and IPA/water as the coagulant.

FIGS. 11A to 11F depict the influence of different coagulant on the plasma recovery and the red blood cell retention of the membranes. Surprisingly, the plasma recoveries of the membranes made from IPA/water and NMP/water as the coagulants were even lower than the membrane made from water as the coagulant. The plasma recovery of a membrane may not only relate to the pore size of the selective layer but also corresponds to the affinity between the membrane and the absorbent and the spreading of blood on the bottom surface of the membrane. Membranes produced from a coagulant of NMP/water were wrinkled, resulting in an ineffective contact between the membrane and the absorbent. Thus, less plasma could be drawn to the absorbent. Membrane made from a coagulant of IPA/water had a relatively dense bottom surface. As a result, the spreading of blood at the membrane bottom surface might not be as good as the membranes made from NMP/water or water as the coagulants. All membranes fabricated in the sections had almost 100% permeations of amino acids, for instance, glutamine acid, histidine, etc. Based on the membrane morphologies in FIGS. 10A to 10L, the pore size distribution was not uniform for membranes made from a combination of TIPS and NIPS. The pore size of the formed membranes could be in the range of 0.10 to 1.00 μm.

### INDUSTRIAL APPLICABILITY

The multi-layered membrane may be used as a diagnostic device and may be used in a variety of applications such as biosensors and as an extractor of cells or liquids from a sample of body fluid. It may be used as a membrane with tunable permeability in a wide range of applications.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1-27. (canceled)

28. A multi-layered membrane for separating components in an aqueous sample comprising:
a porous layer for separating or retaining at least one component from said aqueous sample therein; and
an absorbent layer comprising a superabsorbent or absorbent material for removing liquid from said porous layer.

29. The multi-layered membrane of claim 28, wherein said porous layer contains pores having an effective pore diameter in the range of 0.1 μm to more than 30 μm, or
wherein said porous layer has a pore density in the range of 40% to 95%.

30. The multi-layered membrane of claim 28, wherein said porous layer is a peelable layer, or
wherein said porous layer is further modified to prevent blood clotting and reduce free radicals.

31. The multi-layered membrane of claim 28, wherein said superabsorbent or absorbent material is selected from the group consisting of sodium polyacrylate, polyacrylic acid, alginic acid, starch, hydroxylethyl starch, modified starch, alpha cellulose, modified cellulose, chitosan, carboxylmethyl cellulose, montmorillonite, polyvinyl alcohol, polyethylene oxide, polyacrylamide, hydrolysed polyacrylonitrile, dextran, carboxylmethyl dextran, carbon nanotubes, silica, cotton, rayon, cellulosic pulp, synthetic pulp, bamboo silk, zeolite, glass fibers, polyester fibers, polyethylene fibers, fleece, and mixtures thereof.

32. The multi-layered membrane of claim 28, further comprising a top layer comprising a peelable matrix layer.

33. The multi-layered membrane of claim 32, wherein said top layer comprises a symmetric or an asymmetric membrane matrix.

34. The multi-layered membrane of claim 32, wherein said top layer comprises a material selected from the group consisting of polyarylonitrile (PAN), polyethersulfone (PES), sulfonated polysulfone (SPSf), sulfonated polyethersulfone (SPES), cellulose acetate (CA), cellulose acetate butyrate, ethylcellulose, hydroxylpropyl cellulose, polyurethane, poloxamer polyols, poly(vinyl alcohol), poly(vinyl chlorine), polytetrafluoroethylene (PTFE), polyvinylidene difluoride (PVDF) and combinations thereof.

35. A method of preparing a multi-layered membrane comprising a porous layer and an absorbent layer, the method comprising the steps of:
(a) providing a dope solution of a porous layer material in a solvent;
(b) casting the dope solution to form the porous layer via a method selected from the group consisting of electrospinning, non-solvent induced phase separation (NIPS), thermally induced phase separation (TIPS), vapor induced phase separation (VIPS), a combination of NIPS and TIPS (N-TIPS), and combinations thereof; and
(c) incorporating the absorbent layer adjacent to the porous layer via physical interaction or chemical treatment, wherein the absorbent layer comprises a superabsorbent or absorbent material for removing liquid from said porous layer.

36. The method of claim 35, wherein:
the porous layer material has a concentration in the range of 3.0 weight % to 10.0 weight %; and
the solvent has a concentration in the range of 90.0 weight % to 97.0 weight %, based on the total weight of the dope solution.

37. The method of claim 35, wherein said porous layer material is selected from the group consisting of polyarylonitrile (PAN), polyethersulfone (PES), sulfonated polysulfone (SPSf), sulfonated polyethersulfone (SPES), cellulose acetate (CA), cellulose acetate butyrate, ethylcellulose, hydroxylpropyl cellulose, polyurethane, poloxamer polyols, poly(vinyl alcohol), poly(vinyl chlorine), polytetrafluoroethylene (PTFE), polyvinylidene difluoride (PVDF) and combinations thereof, or
wherein said solvent is selected from the group consisting of N-methylpyrrolidone (NMP), dimethylformamide (DMF), dimethylacetamide (DMAc), dimethyl sulfoxide (DMSO), hexafluoroisopropanol, and combinations thereof.

38. The method of claim 35, wherein when the method used in casting the dope solution to form the membrane is electrospinning, the time taken to collect the porous layer is in the range of 15 minutes to 120 minutes, or
wherein when the method used in casting the dope solution to form the membrane is electrospinning, the porous layer is collected using a drum roller with a roller speed in the range of 70 rpm to 1000 rpm.

39. The method of claim 35, wherein when the method used in casting the dope solution to form the membrane is selected from NIPS, TIPS or N-TIPS, the porous layer is casted using a casting knife with a height in the range of 50 μm to 500 μm.

40. The method of claim 35, wherein said dope solution in step (a) further comprises an additive.

41. The method of claim 40, wherein said additive is selected from the group consisting of methanol, ethanol, isopropanol, acetone, tetrahydrofuran, water, glycerol, ethylene glycol, and combinations thereof, or
wherein during electrospinning, the weight percent ratio of the solvent and additive is in the range of 100:1 to 3:1.

42. The method of claim 35, wherein during TIPS, a partial dope phase separation through VIPS process occurs, or
wherein during TIPS, the porous layer material is PAN, the solvent is a mixed solvent of DMSO/water at 85/15% by volume, or the porous layer material has a concentration in the range of 40.0 mg/ml to 120.0 mg/ml.

43. The method of claim 35, wherein when using N-TIPS, the casting dope solution is cooled in water at 25° C., or
wherein when using N-TIPS, the porous layer material is PAN, or the porous layer material has a concentration in the range of 3.60 weight % to 6.50 weight % of the dope solution.

44. The method of claim 35, further comprising the step of modifying said porous layer by physical or chemical means to contain specific binding sites for desired molecules.

45. A method of separating blood plasma from a whole blood sample, comprising applying said whole blood sample to a multi-layered membrane, wherein said multi-layered membrane comprises a porous layer and an absorbent layer comprising a superabsorbent or absorbent material for removing liquid from said porous layer.

46. The method of claim 45, wherein said whole blood sample is applied to a bottom surface of the porous layer where there are larger pores of greater than 30 μm pore size as compared to a top surface of the porous layer.

47. A diagnostic device for separation of blood plasma from a whole blood sample, comprising a multi-layered membrane comprising a porous layer and an absorbent layer comprising a superabsorbent or absorbent material for removing liquid from said porous layer.
